(19) United States
(12) Patent Application Publication
(10) Pub. No.: US 2009/0043180 A1
(43) Pub. Date: Feb. 12, 2009

(54) SENSOR AND SYSTEM PROVIDING PHYSIOLOGIC DATA AND BIOMETRIC IDENTIFICATION

(75) Inventors: Gary Tschautscher, Carver, MN (US); Jayant Parthasarathy, Minnetonka, MN (US)

Correspondence Address: SCHWEGMAN, LUNDBERG & WOESSNER, P.A., P.O. BOX 2938, MINNEAPOLIS, MN, 55402, US

(73) Assignee: NONIN MEDICAL, INC., Plymouth, MN (US)

(21) Appl. No.: 11/835,741

(22) Filed: Aug. 8, 2007

(51) Int. Cl. A61B 5/00

(52) U.S. Cl. 600/323

(57) ABSTRACT

A device and method of use combining a non-invasive sensor for measuring a physiologic attribute with a biometric identification means. One embodiment of the device and method includes a sensor that has incorporated therein both an oximeter and a fingerprint sensor. The sensor may be connected to a controller including a fingerprint identification circuit in addition to the oximeter circuit and other physiological circuitries such as ECG, pulse or heart rate, NIBP (Non-Invasive Blood Pressure) and temperature. A display may provide an indication of the measured blood oxygen saturation level along with identification information. The display may be located to provide remote monitoring of oxygen saturation and fingerprint identification of the patient, for example, at a central station. The fingerprint identification circuit may be activated individually to obtain the patient fingerprint, which may be recorded in a memory, either in the controller or a remote memory store, or both.

### TECHNICAL FIELD

The present disclosure relates to a sensor, an apparatus and method for performing non-invasive physiologic measurements. More specifically, the present disclosure is directed to a pulse oximeter having a Fingerprint reader and system of use.

### BACKGROUND OF THE INVENTION

Non-invasive oxygen saturation sensors are well known. Pulse oximetry involves the non-invasive monitoring of oxygen saturation level in blood-profused tissue indicative of certain vascular conditions. Pulse oximetry is typically used to measure various blood flow characteristics including, but not limited to, the blood-oxygen saturation of hemoglobin in arterial blood, the volume of individual blood pulsations supplying the tissue, and the rate of blood pulsations corresponding to each heartbeat of a patient.

### BRIEF SUMMARY OF THE INVENTION

The present invention is directed to a device and method that combines a non-invasive sensor for measuring a physiologic attribute with a biometric identification means. To provide accurate identification of a patient, one embodiment of the device and method of the present invention includes a portable sensor that has incorporated therein both an oximeter and a fingerprint sensor. The sensor may be connected to a controller including a fingerprint identification circuit in addition to the oximeter circuit and other physiological circuitries such as ECG, pulse or heart rate, NIBP (Non-Invasive Blood Pressure) and temperature. A display may provide an indication of the measured oxygen saturation level of the blood of the patient along with identification confirmation. The display may be remotely located to provide monitoring of oxygen saturation and fingerprint identification of the patient, for example, at a central station. The fingerprint identification circuit may be activated individually to obtain the fingerprint of a patient, which may be recorded in a memory, either in the controller or a remote memory store, or both.

The controller may include a communications port that enables it to be connected to a remote storage facility, for example the patient record repository. The fingerprint image of a patient may be obtained and stored in the memory store of the remote computer, for identifying the patient and matching the patient records and other being measured physiological data, which may also be stored in the memory of the remote computer. The connection of the sensor to a remote controller may be by way of a conventional cable or short range wireless communications protocol, such as Bluetooth. As a result, the sensor does not need to be physically connected to the controller. Portions of the controller can be provided within the sensor housing. In one embodiment, the controller and display are provided within a portable finger-mounted sensor.

An embodiment of the present invention provides an physiologic measurement circuit to determine, for example, oxygen saturation and a biometric identification circuit to identify the patient, so that the measured physiological data can be correlated or matched to the patient.

The foregoing has outlined rather broadly the features and technical advantages of the present invention in order that the detailed description of the invention that follows may be better understood. Additional features arid advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also he realized by those skilled in the art that such equivalent constructions do not depart from the spirit and scope of the invention as set form in the appended claims. The novel features which are believed to be characteristic of the invention, both as to its organization and method of operation, together with further objects and advantages will be better understood from the following description when considered in connection with the accompanying FIGS. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1A is a perspective view of a pulse oximeter according to one illustrative embodiment;

FIG. 1B is a partially exploded view of the pulse oximeter according to one embodiment; and

FIG. 2 is an exploded view of the pulse oximeter according to one illustrative embodiment.

FIG. 3 is a perspective view of another embodiment of a pulse oximeter according to present invention.

FIG. 4 is a flow chart of an exemplary method of the present invention.

### DETAILED DESCRIPTION OF THE INVENTION

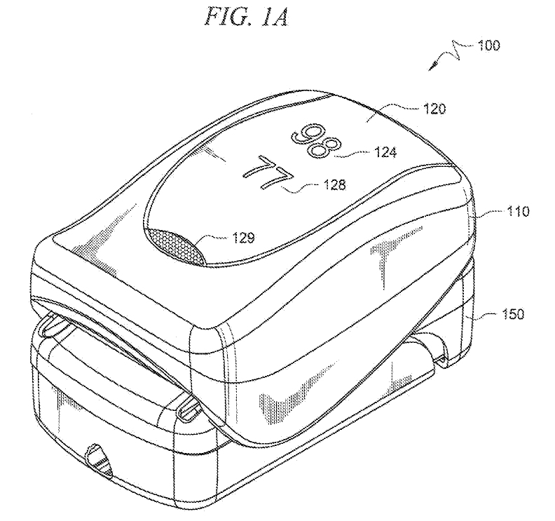

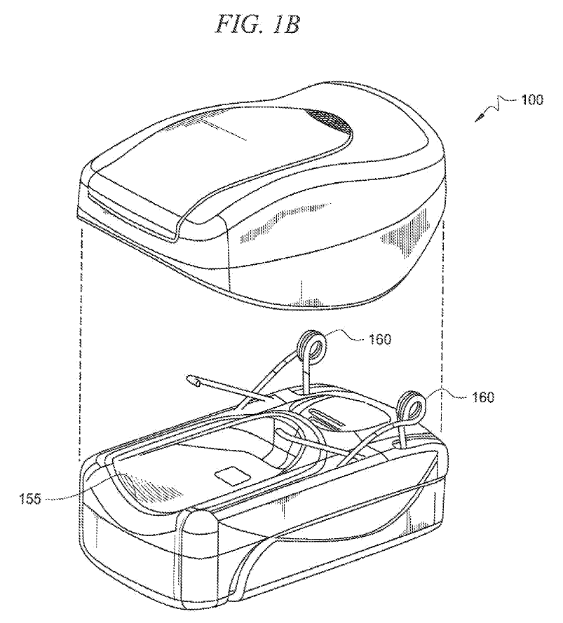

FIG. 1 is a perspective view of a pulse oximeter 100 according to one illustrative embodiment. FIG. 1B is a partially exploded view of pulse oximeter 100. For purposes of this discussion FIGS. 1A and 1B will be discussed together. In one embodiment, the pulse oximeter 100 measures the oxygen saturation of a patient utilizing known oximetry techniques. One type of pulse oximeter sensor is disclosed in U.S. Pat. No. 5,800,349, to Isaacson et al., incorporated by reference herein. However, in other embodiments the pulse oximeter 100 can include the capability to detect and/or capture carbon monoxide levels, ECG waves, pulse or heart rates and temperature. While the present discussion proceeds with respect to a pulse oximeter those skilled in the an will recognize that a variety of devices may be used to collect a physiologic value of the patient.

Pulse oximeter 100 includes a fingerprint reader 270 to obtain a fingerprint image of a patient or care-giver or both. Fingerprint reader 270 and its operation will be described in more detail hereinafter. In one embodiment, functions of the pulse oximeter 100 are controlled or limited by an obtained fingerprint image. For example, access to medical records for storage or review of medical records may require confirmation of a fingerprint image with a reference image.

Aspects of pulse oximeter 100 include a housing having a top portion 110 and a bottom portion 150. In this embodiment the pulse oximeter is configured to measure the blood oxygenation level by accessing a portion of a phalange (such as a finger or toe) of the body. However, other parts of the body can be used. The top portion 110 and bottom portion 150 are, in one embodiment, hinged together such that relatively constant pressure is applied to a finger when it is inserted into the pulse oximeter. However, in other embodiments, the pulse oximeter housing can be flat and not hinged. The top portion 110 includes a display 120. The display 120 is configured to display information related to the detected oxygen levels in the blood. The display 120, in one embodiment, uses light emitting diodes (LED) to display the information. However, other types of displays can be used such as LCD. Display 120 provides a visual indication of, in one embodiment, detected oxygen saturation range (% SpO₂) and the detected pulse rate (beats per minute). These are illustrated on the display 120 at lines 124 and 128 respectively. However, other information can be displayed on display 120. In another embodiment, display 120 could provide detected blood pressure information.

The bottom portion 150, in one embodiment, includes a curved portion 155 and a pair of spring arms 160. The curved portion 155 is shaped to accept the bottom portion of a finger. In one embodiment, the curved portion 155 is shaped such that it can accommodate a range of finger thicknesses. Typical finger thicknesses can range, for example, from 8 mm to 26 mm. This corresponds to the size of a pediatric (child) finger to that of an average adult finger. However, depending on the needs of the pulse oximeter 100, other sizes and shapes can be used for the curved portion.

The spring arms 160 are provided to hold the top portion and the bottom portion together such that sufficient pressure is applied to the finger to generate an acceptable reading for the photoplethysmographic process. However, in alternative embodiments, a single spring arm or other method of biasing the top portion 110 and bottom portion 150 together can be used. In one embodiment, the spring arms are made from metal. However, other materials can be used for the spring arms. The spring arms 160 are arranged such that they permit the pulse oximeter 100 to hinge or open to accept the finger. The spring arms are biased towards the closed position such that the pulse oximeter 100 tends to stay in the closed position when not in use.

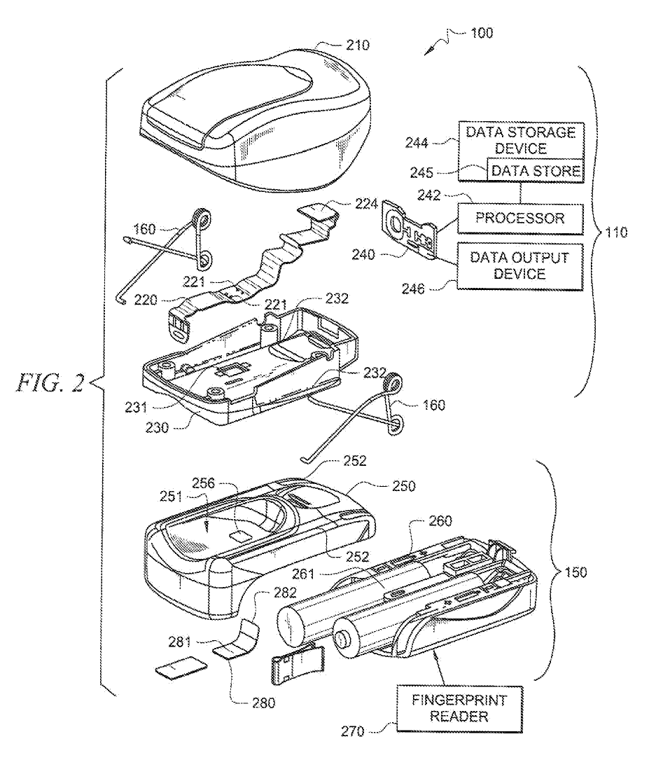

FIG. 2 is an exploded view of pulse oximeter 100 according to one illustrative embodiment. Both the top portion 110 and the bottom portion 150 are divided into a number of parts. Top portion 110 is illustrated having a top casing 210, a sensor strip 220, a bottom casing 230 and a circuit board 240. However, other components can be present. Top casing 210 is simply the outer covering of the top portion 110. The top casing can include the display 120. Depending on the design, the top casing can take a variety of shapes, but is typically shaped to promote easy use of the pulse oximeter. The bottom casing 230 forms the underside of the top portion 110. The bottom casing 230 is, in some embodiments, curved to correspond to the shape of a finger. This curvature assists in allowing the LEDs to be closer to the finger, and allows for a more comfortable test. However, in other embodiments, the bottom casing can be flat. The bottom casing 230 includes a cutout or aperture 231 that corresponds to the location of the LEDs on the sensor strip 220. In some embodiments, the aperture 231 may include a transparent cover to protect the LEDs 221 from debris or other contaminants that may be present. Bottom casing 230 also includes apertures 232 that permit the spring arms 160 to interface with the top portion 110.

The sensor strip 220 includes, in one embodiment, LEDs 221 for use during the oximetry process. However, other illumination components can be used. In one embodiment, LEDs 221 include two LEDs, one LED emitting red light having a wavelength of 660 nm, and a second LED emitting infrared light having a wavelength of 910 nm. However, other wavelengths that produce red and infrared light can be used. In alternative embodiments where the pulse oximeter can detect CO levels in the blood additional LEDs are present. These additional LEDs operate at different wavelengths and thus emit different colors of light than the LEDs used to detect oxygenation in blood. The sensor strip 220 also includes a wire or other electrical connection to transmit signals to/from the circuit board 240. In some embodiments, the strip 220 can also include, either with or in place of, the LEDs 221 the photodiodes necessary to perform a photoplethystnographic process.

Circuit board 240, in one embodiment, is a small hoard that processes the received signals from the photodiodes 281 and the fingerprint reader 270. The circuit board 240 may include a processor 242 to process the received signals using, for example, a photoplethysmographic process. The processor 242 can be any processor capable of analyzing the received signals. The processor 242 analyzes the received signal and generates an output that is transmitted to the display component 120. This output can be transmitted over electrical connection 224 on the sensor strip 220. In one embodiment, the circuit board includes a data storage device 244. The data storage device 244 can be any type of data storage device such as flash memory or a disc drive. In some embodiments, the data storage device 244 can be a removable storage media. When the data storage device 244 is removable, the circuit board 240 can include an interface to accept or communicate with the media.

The data storage device 244 illustratively includes a data store 245. The data store 245 stores data related to pulse oximeter measurements. This information can be stored as a table of data. However, other methods of storing data can also be used. The table of data can be stored using any method, such as, for example, sequential query language (SQL) or extensible mark-up language (XML). In some embodiments, the circuit board also includes a connection to a data output device 246. This data output device permits the transmission data in the data store to an outside computing device. The data output device 246 can be located on either the top portion 110 or the bottom portion 150. Further, the data output device can be any device that permits the transmission of information from the pulse oximeter 100 to the outside computing device, such as USB, Firewire, Bluetooth, IR, etc. This data can be further protected from unauthorized access by using the fingerprint reader 270.

The bottom portion 150 is illustrated having a top casing 250, a bottom casing 260, a fingerprint reader 270, and a sensor strip 280. The top casing 250 includes a finger rest area 251 and an aperture 256. The finger rest area 251 is shaped to receive a bottom part of a finger. The aperture 256 is located in a portion of the finger rest area 251. The location of the aperture is preferably at the point where the tip of the finger extends slightly beyond the aperture during testing. The aperture 256 is sized such that a significant portion of the finger tip is exposed to the photodiodes. Again a transparent cover may be provided to protect the photodiodes from debris. In some embodiments, the aperture 256 is sized to accommodate LEDs that are received by the photodiodes 221. Also included in the top casing 250 are apertures 252 that permit the spring arms 160 to interface with the bottom portion 150.

The bottom casing 260 provides, in one embodiment, a housing for a power supply 261 used to power the pulse oximeter 100. In one embodiment, the power supply is two AA batteries. However, other types of power supplies can be used. Also included in the bottom casing 260 in one embodiment, is the fingerprint reader 270. However, the fingerprint reader can be located in other areas instead.

Sensor strip 280 includes photodiodes 281, and electrical connection 282. The photodiodes 281 are arranged to receive light signals from the LEDs 221 located on the sensor strip 220 in the top portion 110. In one embodiment, the photodiodes 281 receive both red and infrared light that has passed through the finger. This received light causes the photodiode 281 to generate a signal. This signal is passed along electrical connection 282 to the circuit board 240 for photoplethysmographic processing to occur. Electrical connection 282 can be any electrical connection such as wire or etched paths into a surface. In alternative embodiments, the LEDs can also be on the strip 280 either alone or in conjunction with the photodiodes 281.

Fingerprint reader 270 is located, in one embodiment, on the outside of the bottom portion 150 of the pulse oximeter 100. However, in other embodiments, the fingerprint reader 270 can be located on the outside of the top portion 110, or the sides of either the top or bottom portion. The fingerprint reader operates in conjunction with or separate from the pulse oximetry process. Depending on the location of fingerprint reader 270 some components of the pulse oximeter may change their respective configurations. The fingerprint reader 270 can use one of a number of approaches in obtaining an image of a fingerprint.

Fingerprint reader 270, in one embodiment, is an optical system for detecting and analyzing a fingerprint. In this embodiment, the reader includes a device for capturing an image of the fingerprint, and a device for illuminating the fingerprint. In one embodiment, the image device is a charged coupled device (CCD) camera. The CCD camera includes an array of light sensitive diodes or photosites. To illuminate the fingerprint the device uses, in one embodiment, an array of LEDs that highlight the ridges and valleys of the fingerprint.

In another embodiment, the fingerprint reader 270 is a capacitive sensor. Instead of using light to generate the image of the fingerprint the capacitive sensor uses capacitors and electrical current to generate an image of the ridges and valleys of the fingerprint. The capacitive sensor includes a number of cells. Each of the cells includes two conductor plates that are covered with an insulating layer.

In another embodiment, the fingerprint reader 270 is a surface pressure sensor. In this embodiment the surface pressure sensor uses a piezoelectric surface array to generate an image of the fingerprint. The surface ridges of the fingerprint contact the surface array and are used to generate the image. The surface pressure sensor generally has a larger sensing area than other types of fingerprint sensors, but tends to have a lower image quality.

In yet another embodiment, the fingerprint reader 270 is an E-field sensor. The E-field sensor allows the fingerprint reader to image the fingerprint below the surface layer. This allows for the reader to obtain a better result (or image) regardless of the condition of the patient's finger. The E-field sensor includes an antenna array, at least one semiconductor, and a under-pixel amplifier. The semiconductor generates a field by forcing a small electrical current through the finger. This generated field mimics the epidermal layer of the fingerprint. That is, the field is representative of the layer below the surface of the skin. This field is read by the antenna array, which detects the generated linear field below the surface of the skin. This information is processed by the under-pixel amplifier to generate an image of the fingerprint.

In other embodiments of oximeter 100, fingerprint reader 270 may incorporate an electro-optic sensor, RF field sensor, tactile MEMS sensor, thermal sensor, ultrasound sensor, sweep type sensor.

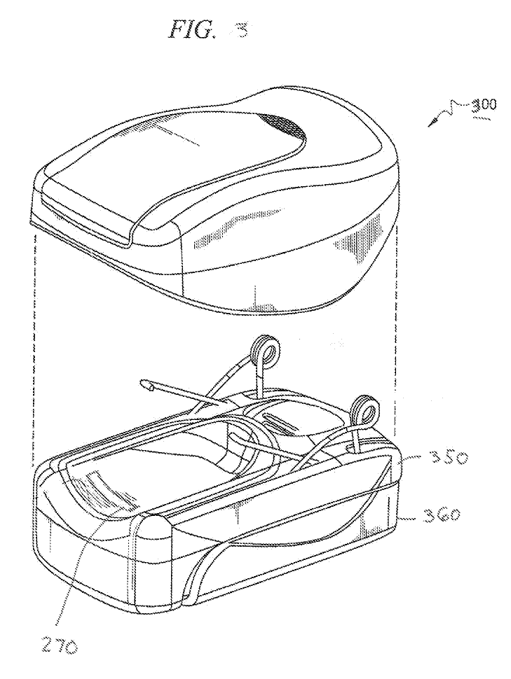

FIG. 3 illustrates another embodiment of pulse oximeter 300 having fingerprint reader 270 located on an upper surface of the top casing 350 of the bottom portion 360. In such an embodiment 300, a “sweep-type” fingerprint reader 270 would, capture the fingerprint as the user inserts the finger into oximeter 300.

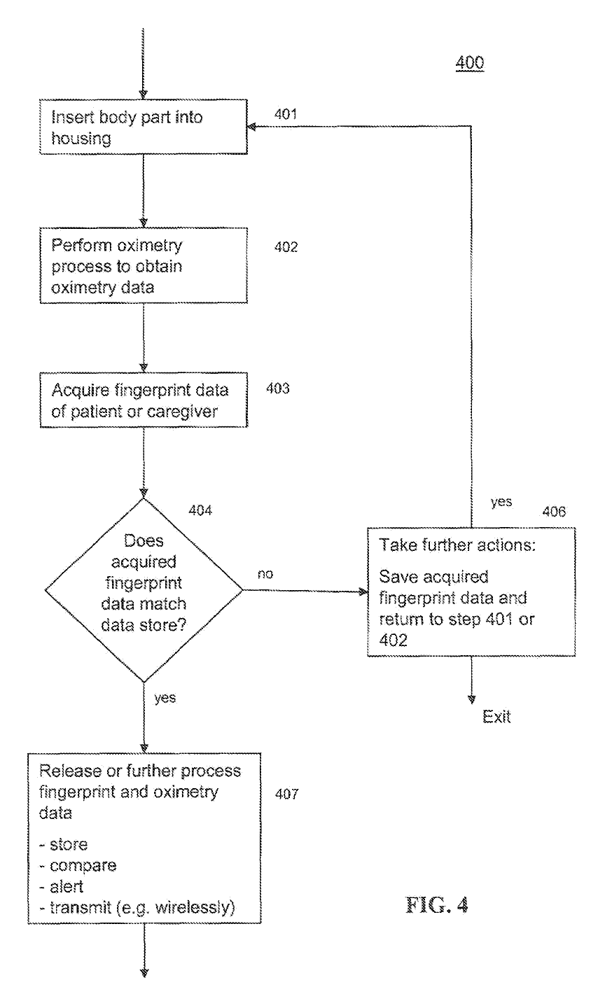

FIG. 4 is a flow chart 400 of operations of an exemplary embodiment of the present invention. At step 401, a patient body part is inserted in the housing of oximeter 100. An oximetry process is performed at step 402 using a light emitter and light detector to determine oximetry data of the patient. Fingerprint data is acquired at step 403 using fingerprint reader 270. At step 404, a comparison is made between the acquired fingerprint data from step 403 and previously stored fingerprint data. If a match is determined, oximetry data can be released at step 405 for further processing or exportation. If no match is determined at step 404, step 406 permits fingerprint data to be stored for subsequent use prior to returning to step 401. Fingerprint data acquired at step 403 may be related to the patient or caregiver or both.

Regardless of which type of fingerprint reader is used for the fingerprint reader 270 the result is a generated image of the associated fingerprint. Generally speaking, the patient or caregiver places one finger on, over or through fingerprint reader 270. Depending on the configuration of the system the actual image generation can occur either at the fingerprint reader 270 or at the processor 242. In one embodiment, this image is provided to the processor 242. However, in other embodiments, fingerprint reader 270 merely provides the data necessary for processor 242 to generate the image.

In one embodiment, processor 242 receives the fingerprint image, and performs at least one operation using the image. The specific operation executed is dependent on the configuration of the pulse oximeter 100. In one embodiment, the generated image is stored in the data store along with the associated oxygenation levels and pulse rate. This enables the fingerprint to be associated with a given set of patient data. In other embodiments, additional information can be stored in the data store at this time such as a date and time that a reading was taken. This stored information can then later be downloaded to a central database and added to the appropriate patient record. Thus, a nurse or other medical practitioner can sample a number of patients using the same device without having to write down the results immediately. Further, associating the patient's fingerprint with the data reduces the risk of incorrect information being associated with the patient.

Fingerprint reader 270 may be connected to a remote computer and be used to sense the fingerprint of the patient, so that the identity of the patient is preestablished in the remote computer. By thus preestablishing the identify of a patient, as the patient's physiological data is collected by oximeter 100, the data collected and processed by processor 242 could readily be routed to the remote computer and matched to the patient for storage and analysis remotely from the patient.

In identifying a fingerprint the fingerprint reader 270 or the processor 242, in one embodiment, uses minutiae-based matching. However, in other embodiments, global pattern matching can be used.

In another embodiment, the fingerprint image can be used to search the data store 245 for previous entries. All or portions of fingerprint data store 245 may be located remote from said oximeter 100. If a match between the detected fingerprint image and a stored fingerprint image are found in the data store, the processor can compare the associated oxygenation levels and pulse rates. If the comparison results are outside an acceptable margin, (e.g., 5%) the pulse oximeter can generate an indication to the user alerting them of a possible problem. In yet another embodiment, the fingerprint can associate the administrator of the photoplethysmographie process to the results, thus providing a form of quality assurance.

In another embodiment, the fingerprint reader 270 is used to protect the data in data store 245. In today's medical practices patient security and privacy are a major concern so it is necessary to secure the information in the pulse oximeter 100 in the event it is lost or stolen. In this protective embodiment, the fingerprint reader 270 is used to verify that a person attempting to remove data from the data store is authorized to do so. Prior to permitting data in the data store to be downloaded via the data output device 246, the user is asked to provide their fingerprint. The fingerprint is read at the fingerprint reader 270 and compared against a list of authorized users. Preferably this list of authorized users is stored in the data store 245. However, this list can be located in other locations such as on the remote computing device. If there is a match between the fingerprint and the list then the data can be offloaded. If there is not a match then the oximeter 100 will not permit the data to be offloaded.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A sensor system comprising:
a light emitter and a photodetector disposed on an inside portion of a housing, said photodetector receiving light from said emitter after passing through a portion of a patient;
a fingerprint reader, with at least a portion thereof being disposed on the housing, said reader providing fingerprint data of a patient or care giver; and
a processor for determining patient oximetry data based on said received light and releasing said data upon satisfaction of a condition relating to said fingerprint data.

2. The device of claim 1 wherein the processor performs a blood oxygen saturation calculation and a fingerprint image comparison.

3. The device of claim 2 further comprising:
a data storage device communicatively connected to the processor.

4. The device of claim 3 further comprising:
a communications device configured to transmit data from the data storage device to a remote location.

5. The device of claim 1 further comprising:
a display component on said housing and configured to display an oxygen saturation level.

6. The device of claim 1 wherein the fingerprint reader includes one or more of an optical sensor, a capacitive sensor, an E-field sensor, an electro-optic sensor, an RF field sensor, a tactile MEMS sensor, a thermal-sensor, an ultrasound sensor, a sweep type sensor and a surface pressure sensor.

7. The device of claim 1 wherein the device is configured to detect blood oxygen saturation or carbon monoxide in a bloodstream.

8. The device of claim 1 wherein the processor releases the oximetry data when a match is detected between the patient and a stored fingerprint data.

9. The device of claim 1 wherein the processor releases the oximetry data when a match is detected between the care giver and a stored fingerprint data.

10. A sensor system method comprising;
performing an oximetry process on a portion of a body part of a patient including emitting light from a light emitter and detecting light passing through said body part portion, said process yielding oximetry data;
acquiring a fingerprint image;
determining whether the fingerprint image matches a stored fingerprint image; and
storing said oximetry data in association with said stored fingerprint image so as to provide a record of oximetry data of said patient.

11. The method of claim 10 further comprising:
comparing oximetry data with a previously stored data of the patient.

12. The method of claim 11 further comprising:
generating an alert if a difference between the oximetry data and the previously stored data exceeds a predetermined threshold.

13. The method of claim 10 further comprising:
exporting the stored results and the image to a remote computing device.

14. The method of claim 13 further comprising;
obtaining an image of a second fingerprint and comparing the image of the second fingerprint with a list of approved fingerprints; and
exporting the oximetry data only if the comparing identifies a match in the list.

15. A sensor system method comprising:
acquiring fingerprint data of multiple patients or a care givers of the patients and storing said fingerprint data in records of said multiple patients;
determining oximetry data of one of said multiple patients using a light emitter and light detector controlled by a processor;
acquiring fingerprint data of said one of said multiple patients or a care giver of said one of said multiple patients;
comparing said acquired fingerprint data with fingerprint data stored in said records; and
based on said comparing, performing an action on said acquired fingerprint data.

16. The method of claim 15 wherein said performing an action includes storing oximetry data of said patient.

17. The method of claim 16 wherein said performing an action includes comparing said stored oximetry data against previously stored data.

18. The method of claim 17 wherein said performing an action includes alerting the patient or care giver when said comparing yields a difference exceeding a predetermined threshold.

19. The method of claim 15 wherein said comparing is performed at a remote site away from the patient after said fingerprint data is transmitted to said remote site.

20. The method of claim 19 wherein said fingerprint data and oximetry data is wirelessly transmitted from a housing proximate to the patient.
